SEQUENCE LISTING (6 sequences)

SEQ ID NO: 1 - DNA, 8886 bp, Homo sapiens
aagcttcgaagtttaaactgagtgccgccaccatgaccctgcggctgctggtggctgccctgtgtgctggcatcctggccgaggctcctagagtgcgggcccagcaccgcgagagagtgacctgcaccagactgtacgccgccgatatcgtgttcctgctggacggctcctcctccatcggccggtccaacttccgggaagtgcggtccttcctggaaggcctggtgctgcctttctccggcgctgcctctgcccagggcgtcagattcgccaccgtgcagtactccgacgacccccggaccgagttcggcctggatgctctgggctctggcggcgacgtgatccgggccatcagagagctgtcctacaagggcggcaacacccggacaggcgccgctatcctgcacgtggccgaccatgtgtttctgccccagctggccagacccggcgtgcccaaagtgtgcatcctgatcaccgacggcaagtcccaggacctggtggacaccgccgctcagagactgaagggccagggcgtgaagctgttcgccgtgggcatcaagaacgccgaccccgaggaactgaagcgggtggcctcccagcctacctccgatttcttcttcttcgtgaacgacttctccatcctgcggaccctgctgcccctggtgtctcggagagtgtgtaccaccgctggcggcgtgccagtgacccggcctcctgacgattctacctccgcccctcgggatctggtgctgtccgagccttccagccagtccctgagagtgcagtggaccgccgcctctggccccgtgaccggctacaaggtgcagtacacccctctgaccggcctgggccagcctctgccttctgagcggcaagaagtgaacgtcccagccggcgagacatccgtgcggctgagaggcctgaggcccctgaccgagtaccaagtgaccgtgatcgccctgtacgccaacagcatcggcgaggccgtgtccggcaccgccagaaccacagccctggaaggacccgagctgaccatccagaacaccaccgcccactccctgctggtcgcttggagatctgtgcctggcgccaccggctatcgcgtgacctggcgagttctgtctggcggccctacccagcagcaagagctgggccctggacagggctctgtgctgctgagggacctggaacccggcaccgactacgaagtgacagtgtccaccctgttcggcagatccgtgggccctgccacctctctgatggccagaaccgacgcctccgtggaacagaccctgaggcctgtgatcctgggccccaccagcatcctgctgagctggaacctggtgcccgaggccagaggctaccggctggaatggcggagagagacaggcctggaacctccccagaaggtggtcctgccctccgacgtgaccagataccagctggatggcctgcagcctggcaccgagtacagactgaccctgtacaccctgctcgagggccacgaggtggccacccctgctacagtggtgcctaccggccctgagctgcccgtgtcccctgtgaccgatctgcaggccaccgagctgcctggccagcgcgtcagagtgtcttggtccccagtgccaggcgctacccagtaccggatcatcgtgcggtccacacagggcgtggaaagaaccctggtgctccccggctcccagaccgccttcgacctggatgatgtgcaggccggcctgagctacaccgtgcgggtgtccgctagagtgggccctagagaaggctccgccagcgtgctgaccgtgcgcagagagcctgaaacccctctggccgtgcccggactgcgggtggtggtgtctgatgccaccagagtcagagtggcctggggccctgtgccaggggcctccggcttcagaatctcctggtccaccggctctggccctgagtcctctcagaccctgccccctgactccaccgccaccgatatcaccggactgcagcccggaaccacctaccaggtggccgtgtctgtgctgaggggcagagaagagggcccagccgccgtgatcgtggccaggacagatcctctgggcccagtgcggaccgtgcacgtgacccaggccagctccagctccgtgaccatcacctggaccagagtccctggcgctacaggctacagagtgtcctggcactctgcccacggccccgagaagtcccagctggtgtctggcgaggccaccgtggctgaactggacggcctcgagcccgacacagagtacacagtgcacgtgcgcgcccatgtggctggcgtggacggacctcctgcttccgtggtcgtgcgcaccgctcctgagcccgtgggaagagtgtcccggctgcagatcctgaacgcctccagcgacgtgctgcggatcacctgggtcggagtgaccggcgctaccgcttacagactggcttggggcagatctgagggcggacccatgcggcatcagatcctgcctggcaacaccgactccgccgagatcagaggactggaaggcggcgtgtcctactctgtgcgcgtgaccgccctcgtgggcgacagagaaggcacccccgtgtccatcgtggtcaccacccctccagaggcccctccagctctgggcaccctgcatgtggtgcagcggggcgagcactccctgagactgagatgggagcctgtgcctcgggcccagggcttcctgctgcattggcagcctgaaggcggccaagagcagtctagggtgctgggccccgagctgtccagctaccacctggacggactggaaccagccacccagtacagagtgcggctgtccgtgctgggacctgccggcgagggaccttctgccgaagtgaccgccaggaccgagtcccctcgggtgccctccatcgagctgagagtggtggataccagcatcgacagcgtgaccctggcctggacccctgtgtcccgggcctcttcctacatcctgtcttggaggcctctgaggggcccaggccaagaggtgccaggctcccctcagacactgccaggcatcagctcctcccagcgcgtgacaggactcgagcctggggtgtcctacatcttctccctgacccccgtcctggacggcgtgcgcggacctgaggcttctgtgacccagaccccagtgtgccccagaggcctggccgacgtggtctttctgcctcacgccacccaggacaacgcccacagagccgaggctaccagacgggtgctcgagagactggtgctggccctgggaccactgggcccacaggctgtgcaagtgggcctgctgtcttactcccaccggccctcccccctgttccccctgaacggctctcacgacctgggcatcatcctgcagcggatccgggacatgccctacatggacccctccggcaacaacctgggcaccgccgtggtcacagcccaccggtacatgctggcccccgatgctcctggcagacggcagcatgtccccggcgtgatggtgctgctcgtggacgagcccctgcggggcgacatcttcagccctatcagagaggcccaggctagcggcctgaacgtggtcatgctgggcatggctggcgccgaccctgagcagctgagaaggctggcccctggcatggactccgtgcagaccttctttgccgtggacgacggccccagcctggatcaggctgtgtctggcctggctaccgccctgtgtcaggcctccttcaccacccagcccagacccgagccttgccccgtgtactgccctaagggccagaagggcgagcccggcgagatgggcctgagaggacaagtgggacctccaggcgatcccggcctgcctggaagaacaggcgctcctggacctcagggccctcctggctctgctaccgctaagggcgagagaggcttcccaggcgccgacggcagacctggctctccaggcagagccggcaatcctggaacacctggcgccccaggcctgaagggatctcctggcttgcctggccctaggggcgaccctggcgaaagaggacctagaggccctaaaggcgagccaggcgcccctggccaagtgatcggcggagaaggacctggactgcccggcagaaagggcgatcctggcccttctggcccacccggcccaagaggtcctctgggagatcctggaccaaggggcccaccaggcctgcccggaacagctatgaagggcgataagggcgacaggggcgagcggggaccaccaggaccaggcgaaggtggaatcgctcccggcgaacctgggctgccaggactgcctggatctcccggaccacagggacctgtgggcccacctggcaagaagggggagaaaggcgactccgaggacggggctccaggattgcccggccaaccaggctctcctggcgaacagggtcccagaggacctcccggcgctatcggcccaaagggggacagaggattccctggcccactgggcgaggctggcgaaaaaggcgaacgcggaccccctggccctgccggcagtagaggacttcctggcgttgccggcagaccaggcgccaagggacctgaaggccctccaggccctaccggaaggcagggcgaaaagggggaacctggcaggccaggcgacccagctgttgtgggaccagccgtggctggacccaaaggcgagaaaggggatgtgggacccgctgggcctagaggcgctactggcgttcagggggaaagaggcccccctggactcgtgctgcctggggatccaggtcctaagggggatcccggcgatagaggcccaatcggcctgaccggcagagctggtccccctggcgattccggtcctcccggggaaaaaggggaccccggtagaccaggtcccccaggccctgttggccctcgcggaagagatggcgaagtgggagaaaagggcgacgaaggacccccaggggacccaggacttccaggcaaggctggggagagaggactgaggggcgctccaggtgtcagaggccctgtcggcgagaagggggatcagggcgatccaggcgaggacggcagaaacggctcccctggctctagtggtccaaaaggcgaccggggagagcctgggcctcctgggccaccaggcagactggtcgataccggacctggggccagagagaagggcgaaccaggggataggggccaagaaggcccacgaggaccaaagggcgacccaggattgcctggcgctcctggcgagaggggcatcgagggctttagaggtccacccggtccccaaggcgaccccggcgttaggggacctgctggggagaagggcgacagaggcccacccggactggacggcagatctggcctggatggcaagcctggcgccgctggcccatctggacctaacggcgctgctggcaaagccggggaccctggacgagatggactgccagggctgcggggagaacagggccttccaggaccttcaggaccacctggcctccctggcaagcccggggaggatggaaagcccggcctgaatggaaaaaacggggaacccggggatcctggggaggacggacgcaagggggaaaagggcgattccggcgcctctggcagagagggcagggacggaccaaaaggggagcgcggagcacccggcattctgggtcctcaggggccacctggattgccaggtccagttggtcctcctggccaggggtttcccggcgtcccaggcggtacagggcctaaaggggatagaggcgagacaggcagcaaaggggaacaggggctcccaggcgaaaggggcttgagaggcgagcctggctccgtgcctaacgtggacagactgctggaaaccgccggcatcaaggcctccgccctgcgcgagatcgtggaaacctgggacgagtcctccggctccttcctgcccgtgcctgagcgcagaaggggcccgaaaggggactctggcgagcaaggaccacccggcaaagagggacccatcggcttccctggggagcgggggttgaaaggcgataggggagatccaggcccacaagggcctccagggctggcacttggagagcgtggtcctccaggaccaagcggactggcaggggagcccggaaagcctggaatccccgggttgcctggtagagccggcggagtgggcgaagcaggcaggcctggggaacgcggagagagaggcgaaaagggcgaaagaggggagcagggccgcgacggtccccccggactacctggaactccagggcccccaggaccccccggacctaaggtgtccgtggatgagcctggccccggactgagcggagaacaaggtccacctggcttgaagggtgccaagggggagccaggctctaacggcgatcaagggcccaagggggatcggggagtgcctggcatcaaaggggaccggggcgaacccggtcctagagggcaagacggaaaccccggcttgccgggcgaacggggaatggctggtcccgagggaaagccaggcttgcagggacctagggggcctcccggtcctgtgggtggacatggcgatccgggtccaccaggtgctccaggactcgctggtccagcaggccctcagggaccatccggcctgaaaggggaaccaggcgaaactggccccccaggcagaggcctgacaggccctactggtgctgtgggcctccctggacctcctggccctagtggactcgtgggccctcagggctctcccggactgccaggccaagtgggcgagactggaaaacccggggctcccggcagggatggcgcttctggaaaagacggcgataggggcagccctggcgtgcccggtagtccagggctacctggccctgtgggtcccaaaggggagcctggacctacaggcgcaccaggccaggctgtagtggggctgcctggcgctaaaggcgagaagggtgctcctggcggcctggctggcgatctcgttggagaacctggcgccaagggcgaccgtggcttgccaggacctcgcggcgagaaaggcgaagctggcagagctggcgagcctggggacccaggcgaagatggccagaaaggcgctcccggccctaagggattcaagggcgatccgggcgtgggcgtgccaggctctccaggtcctcctggaccacccggtgtcaagggcgatttgggccttcctggcctgccaggggcacctggcgtcgtgggctttcctggacagaccggcccacggggagagatgggacagccaggccccagcggagaaagagggctggctggcccgcctggcagggaaggcataccaggcccattggggcctccaggcccacctggatctgtggggcctcctggcgcctctggactgaaaggcgacaaaggcgatcctggtgtcggcctgccaggcccaagaggcgagaggggagagcccggcatcaggggcgaagatggacggcctggccaagagggccctcggggattgaccggccctcctggatccagaggcgaacggggggagaagggggacgtgggctctgctggcctcaaaggcgacaagggggactccgccgtgattctgggccctcccggacctcggggagctaagggggacatgggagagaggggtccacggggactggatggggacaagggaccacgcggagacaacggcgacccgggggataagggctccaagggcgaacctggcgataagggatccgctggactgcctggcctgaggggcctgctgggacctcaaggacaaccaggcgccgcaggcatccctggcgaccctggatctcctggaaaggacggcgtgcccggcatccgcggagaaaagggggatgtcggcttcatgggccccagggggctgaagggggaaaggggagtgaagggcgcttgcggcctcgatggggaaaagggggacaagggggaggctggccctccaggacgacctggactggctggccacaagggcgaaatgggagagccaggcgtgcccggacagtccggcgctccaggcaaagagggcctgatcggccccaaaggcgatagaggatttgacggccagcctggcccaaagggcgatcaaggcgaaaaaggggagagaggcacccccggcatcggcggctttccaggcccctctggaaacgatggctctgccggcccacctgggccacctggtagtgtgggaccaagaggccccgagggactgcagggacagaaaggcgagagagggccccctggcgagagagttgtgggagcacctggcgttcccggcgcacccggcgaaaggggagaacaaggcagacctggaccagccggaccccgtggggaaaaaggcgaggccgccctgaccgaggacgacatcagaggcttcgtgcggcaagagatgtcccagcactgcgcctgtcagggccagtttatcgcctccggcagcagacccctgccttcctacgctgccgataccgccggctctcagctgcacgctgtgcctgtgctccgggtgtcccacgccgaggaagaggaaagagtccctcctgaggacgacgagtacagcgagtactctgagtattccgtggaagagtaccaggatcccgaggccccttgggacagcgacgacccttgctccctgcctctggatgagggctcctgcaccgcctacaccctgagatggtatcaccgggccgtgacaggctccaccgaggcctgtcaccctttcgtgtatggcggctgcggcggcaacgccaatagattcggcacccgcgaggcctgcgagcggagatgtcctcccagagtggtgcagtcccagggcaccggcacagcccaggactgatagtctagagtggccggcc

SEQ ID NO: 2 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 3 - DNA, 1888 bp, Homo sapiens
ccggtgccgggcgaacatggcggcggccaccggaccctcgttttggctggggaatgaaaccctgaaggtgccgctggcgctctttgccttgaaccggcagcgcctgtgtgagcggctgcggaagaaccctgctgtgcaggccggctccatcgtggtcctgcagggcggggaggagactcagcgctactgcaccgacaccggggtcctcttcctccaggagtccttctttcactgggcgttcggtgtcactgagccaggctgctatggtgtcatcgatgttgacactgggaagtcgaccctgtttgtgcccaggcttcctgccagccatgccacctggatgggaaagatccattccaaggagcacttcaaggagaagtatgccgtggacgacgtccagtacgtagatgagattgccagcgtcctgacgtcacagaagccctctgtcctcctcactttgcgtggcgtcaacacggacagcggcagtgtctgcagggaggcctcctttgacggcatcagcaagttcgaagtcaacaataccattcttcacccagagatcgttgagagccgagtgtttaagacggatatggagctggaggttctgcgctataccaataaaatctccagcgaggcccaccgtgaggtaatgaaggctgtaaaagtgggaatgaaagaatatgggttggaaagcctcttcgagcactactgctactcccggggcggcatgcgccacagctcctacacctgcatctgcggcagtggtgagaactcagccgtgctacactacggacacgccggagctcccaacgaccgaacgatccagaatggggatatgtgcctgttcgacatgggcggtgagtattactctgtcgcttccgacatcacctgctcctttccccgcaacggcaagttcactgcagaccagaaggccgtctatgaggcagtgctgctgagctcccgtgccgtcatgggtgccatgaagccaggtgactggtggcctgacatcgaccgcctggctgaccgcatccacctggaggagctggcccacatgggcatcctgagcggcagcgtggacgccatggtccaggctcacctgggggccgtgtttatgcctcacgggcttggccacttcctgggcattgacgtgcacgacgtgggaggctacccagagggcgtggagcgcatcgacgagcccggcctgcggagcctgcgcactgcacggcacctgcagccaggcatggtgctcaccgtggagccgggcatctacttcatcgaccacctcctggatgaggccctggcggacccggcccgcgcctccttccttaaccgcgaggtcctgcagcgctttcgcggttttggcggggtccgcatcgaggaggacgtcgtggtgatcgacagcggcatagagctgctgacctgcgtgccccgcactgtggaagagattgaagcatgcatggcaggctgtgacaaggcctttacccccttctctggccccaagtagagccagccagaaatcccagcgcacctgggggcctggccttgcaacctcttttcgtgatgggcagcctgctggtcagcactccagtagcgagagacggcacccagaatcagatcccagcttcggcatttgatcagaccaaacagtgctgtttcccggggaggaaacacttttttaattacccttttgcaggcaccacctttaatctgttttataccttgcttattaaatgagcgacttaaaatgattgaaaataatgctgtcctttagtagcaagtaaaatgtgtcttgctgtcatttatattccttttcccaggaaagaagcatttctgatactttctgtcaaaaatcaatatgcagaatggcatttgcaataaaaggtttcctaaaatg

SEQ ID NO: 4 - protein, 486 aa, Homo sapiens
MAAATGPSFWLGNETLKVPLALFALNRQRLCERLRKNPAVQAGSIVVLQGGEETQRYCTDTGVLFRQESFFHWAFGVTEPGCYGVIDVDTGKSTLFVPRLPASHATWMGKIHSKEHFKEKYAVDDVQDEIASVLTSQKPSVLLTLRGVNTDSGSVCREASFDGISKFEVNNTILHPEIVECRVFKTDMELEVLRYTNKISSEAHREVMKAVKVGMKEYELESLFEHYCYSRGGMRHSSYTCICGSGENSAVLHGAGAPNDRTIQNGMCLFDMGGEYYCFASDITCSFPANGKFTADQKAVYEAVLRSSRAVMGAMKPGVWWPDMHRLADRIHLEELAHMGILSGSVDAMVQAHLGAVFMPHGLGHFLGIDVHDVGGYPGVRIDEPGLRSLRTARHLQPGMVLTVEPGIYFIDHLLDEALADPARASFLNREVLQRFRGFGGVRIEEDVVVTDSGIELLTCVPRTVEEIEACMAGCDKAFTPFSGPK

SEQ ID NO: 5 - protein, 397 aa, Rattus norvegicus
MIHTNLKKKFSLFILVFLLFAVICVWKKGSDYEALTLQAKEFQMPKSQEKVAMGSASQVVFSNSKQDPKEDIPILSYHRVTAKVKPQPSFQVWDKDSTYSKLNPRLLKIWRNYLNMNKYKVSYKGPGVKFSVEALRCHLRDHVNVSMIEATDFPFNTTEWEGYLPKENFRTKVGPWQRCAVVSSAGSLKNSQLGREIDNHDAVLRFNGAPTDNFQQDVGSKTTIRLMNSQLVTTEKRFLKDSLYTEGILIVWDSYHADIPKWYQKPDYNFFETYKSYRRLNPSQPFYILKPQMPWELWDIIQEISADLIQPNPPSSGMLGIIIMMTLCDQVDIYEFLPSKRKTDVCYYHQKFFDSACTMGAYHPLLFEKNMVKHLNEGTEDYLFGKATLSGFRNIRC

SEQ ID NO: 6 - protein, 247 aa, Homo sapiens
MAHHHHHHLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

(19) United States
(12) Patent Application Publication
(10) Pub. No.: US 2018/0002401 A1
(43) Pub. Date: Jan. 4, 2018

(54) COLLAGEN 7 AND RELATED METHODS

(71) Applicant: PHOENIX TISSUE REPAIR, INC., Andover, MA (US)

(72) Inventors: Mark De Souza, Boston, MA (US); Malini Viswanathan, Acton, MA (US)

(21) Appl. No.: 15/596,447

(22) Filed: May 16, 2017

Related U.S. Application Data
(63) Continuation of application No. 14/236,403, filed on Sep. 12, 2014, now Pat. No. 9,676,837.

(51) Int. Cl. C07K 14/78; C12P 21/02; C12N 9/10; C12N 15/85; C12N 9/02; A61K 38/00; C12N 9/48

(52) U.S. Cl. CPC C07K 14/78; C12Y 304/13009; C12N 9/1048; C12P 21/02; C12N 9/485; C12N 9/1081; C12N 9/0071; A61K 38/00; C12N 2800/22; C12Y 114/11002; C12N 15/85

(57) ABSTRACT

Disclosed are methods of making collagen 7, or functional fragments thereof, as well as collagen 7, and functional fragments thereof produced by such methods, nucleic acids encoding collagen 7, and functional fragments thereof, as well as vectors and host cells comprising such nucleic acids.

The invention relates to collagen 7, collagen 7 related nucleic acids and cells, and related methods.

### BACKGROUND

Collagens are a family of proteins that strengthen and support connective tissues, such as the skin, tendons, ligaments, and bones. Collagen 7, as the major component of anchoring fibrils, functions in strengthening and stabilizing various tissues, including the skin (Ricard-Blum, Cold Spring Harb Perspect Biol 3(1):a004978 (2011)).

Collagen 7 is synthesized as three pro-α1(VII) polypeptide chains, which are subsequently processed and folded into a triple helical procollagen 7 protein in the endoplasmic reticulum. Procollagen 7 is secreted into the extracellular space, where it is further processed into mature collagen 7 (Chung et al. Dermatol Clin 28(1): 93-105 (2010)). Mature collagen 7 undergoes a multistep polymerization process to form the structural anchoring fibrils (Fritsch et al. J Biol Chem 284(44): 30248-30256 (2009)). In the skin, these anchoring fibrils are found in the epidermal basement membrane zone, which is the two-layer membrane located between the top layer of skin, the epidermis, and the underlying dermis. Here the anchoring fibrils connect the epidermal basement membrane to the papillary dermis. This connection aids in holding the epidermal and dermal layers of the skin together, providing structure and stability to the skin (Villone et al. J Biol Chem 283(36): 24506-24513 (2008)).

### SUMMARY OF THE INVENTION

In one aspect, the disclosure features, a method of making collagen 7, or a functional fragment thereof. The method comprises:

providing a cell, e.g., a mammalian cell, e.g., a CHO or HEK cell, genetically modified to express collagen 7, or a functional fragment thereof, and, optionally, one or more polypeptides, e.g., one or more polypeptides that increase collagen 7 production in the cell (e.g., prolidase and/or prolyl hydroxylase); and

culturing the cell under conditions sufficient for the production of collagen 7, or functional fragment thereof, thereby making collagen 7, or a functional fragment thereof.

In one embodiment, the collagen 7 is human collagen 7. In an embodiment, the collagen 7 is encoded by a high glycine codon optimized sequence, e.g., a high glycine codon optimized sequence described herein. In one embodiment, the collagen 7 has the amino acid sequence of SEQ ID NO 2. In one embodiment, the amino acid sequence of the collagen 7 is at least 80, 90, 95, or 99% identical to SEQ ID NO 2. In one embodiment, the amino acid sequence of collagen 7 differs from SEQ ID NO 2 by no more than 1, 2, 3, 4, 5, 6, 7, 8, 9, 10, 15 or 20 amino acid residues.

In an embodiment, the cell is genetically modified to express prolidase, or a functional fragment thereof, and, e.g., the prolidase can be a mammalian, e.g., a human prolidase, or a rodent, e.g., mouse, rat or hamster prolidase. In an embodiment the prolidase is: human prolidase, e.g., human prolidase having the amino acid sequence of SEQ ID NO 4; prolidase having an amino acid sequence at least 80, 90, 95, or 99% identical with SEQ ID NO 4; prolidase having an amino acid sequence that differs from SEQ ID NO 4 by no more than 1, 2, 3, 4, 5, 6, 7, 8, 9, 10, 15 or 20 residues.

In an embodiment, the cell is genetically modified to express prolyl hydroxylase, or a functional fragment thereof, and, e.g., the prolyl hydroxylase can be a mammalian, e.g., a human prolyl hydroxylase, or a rodent, e.g., mouse, rat or hamster prolyl hydroxylase. In an embodiment the prolyl hydroxylase is: human prolyl hydroxylase, e.g., human prolyl hydroxylase having the amino acid sequence of SEQ ID NO X; prolyl hydroxylase having an amino acid sequence at least 80, 90, 95, or 99% identical with SEQ ID NO X; prolyl hydroxylase having an amino acid sequence that differs from SEQ ID NO X by no more than 1, 2, 3, 4, 5, 6, 7, 8, 9, 10, 15 or 20 residues.

In an embodiment, the cell is genetically modified to express a glycosyl transferase, or functional fragment thereof, e.g., a sialyltransferase, or functional fragment thereof. The glycosyl transferase can be a mammalian, e.g., a human glycosyl transferase, e.g., sialyltransferase, or a rodent, e.g., mouse, rat or hamster gylcosyl transferase.

In an embodiment, the glycosyl transferase is a sialyltransferase, e.g., a sialyltransferase having the amino acid sequence of SEQ ID NO 5; a sialyltransferase having an amino acid sequence at least 80, 90, 95, or 99% identical to SEQ ID NO 5; a sialyltransferase having an amino acid sequence that differs from SEQ ID NO 5 at no more than 1, 2, 3, 4, 5, 6, 7, 8, 9, 10, 15 or 20 residues.

In an embodiment, the genetically modified cell comprises a nucleic acid that encodes collagen 7, or a functional fragment thereof, e.g., a high glycine codon optimized nucleic acid sequence, e.g., a nucleic acid sequence of SEQ ID NO 1. In one embodiment, the nucleic acid sequence is at least 80, 90, 95, or 99% identical to SEQ ID NO 1; the nucleic acid sequence differs from SEQ ID NO 1 at no more than 1, 2, 3, 4, 5, 6, 7, 8, 9, 10, 15 or 20 nucleotides. In an embodiment, at least 80, 90, 95, or 99% of the codons have the codon value of SEQ ID NO 1.

In an embodiment the genetically modified cell comprises a nucleic acid that encodes a prolidase, or functional fragment thereof.

In an embodiment the genetically modified cell comprises a nucleic acid that encodes a prolyl hydroxylase, or functional fragment thereof.

In an embodiment the genetically modified cell comprises a nucleic acid that encodes a glycosyl transferase, or functional fragment thereof.

In an embodiment, the cell comprises an expression vector that comprises a nucleic acid sequence that encodes collagen 7, or a functional fragment thereof. In an embodiment said expression vector further comprises a nucleic acid sequence that encodes prolidase, or a functional fragment thereof. In an embodiment said expression vector further comprises a nucleic acid sequence that encodes prolyl hydroxylase, or a functional fragment thereof. In an embodiment, said expression vector further comprises a nucleic acid sequence that encodes glycosyl transferase, or a functional fragment thereof. In an embodiment, said expression vector further comprises a nucleic acid sequence that encodes prolidase, or a functional fragment thereof, and a nucleic acid sequence that encodes glycosyl transferase, or a functional fragment thereof. In an embodiment, said expression vector further comprises a nucleic acid sequence that encodes prolyl hydroxylase, or a functional fragment thereof, and a nucleic acid sequence that encodes glycosyl transferase, or a functional fragment thereof.

In an embodiment, the cell comprises a second expression vector that comprises a nucleic acid sequence that encodes prolidase, or a functional fragment thereof.

In an embodiment, the cell comprises a second expression vector that comprises a nucleic acid sequence that encodes prolyl hydroxylase, or a functional fragment thereof.

In an embodiment, the cell comprises a third expression vector that comprises a nucleic acid sequence that encodes glycosyl transferase, or a functional fragment thereof.

In an embodiment, the cell comprises a second expression vector that comprises a nucleic acid sequence that encodes prolidase, or a functional fragment thereof, and a third expression vector that comprises a nucleic acid sequence that encodes glycosyl transferase, or a functional fragment thereof.

In an embodiment, the cell comprises a second expression vector that comprises a nucleic acid sequence that encodes prolyl hydroxylase, or a functional fragment thereof, and a third expression vector that comprises a nucleic acid sequence that encodes glycosyl transferase, or a functional fragment thereof.

In an embodiment, the cell is a mammalian cell, e.g., a human, or rodent, e.g., a rat, mouse or Chinese hamster cell.

In an embodiment, the cell is a CHO cell.

In an embodiment, the cell is a HEK293 cell.

In an embodiment, the method further comprising recovering collagen 7, or the functional fragment thereof, from said cultured cell.

In an embodiment, the collagen 7, or functional fragment thereof, is recovered from culture medium.

In an embodiment, the method further comprises purifying collagen 7, or functional fragment thereof, from said cultured cell.

In an embodiment, the method further comprising purifying collagen 7, or functional fragment thereof, from culture medium.

In an embodiment, at least 30, 40, 50, 60, 70, 80, 90 or 95% of said collagen 7, or functional fragment thereof, is incorporated into homotrimers.

In an embodiment, at least 30, 40, 50, 60, 70, 80, 90 or 95% of said collagen 7, or functional fragment thereof, is incorporated into hexamers.

In another aspect, the disclosure features, a vector described herein.

In another aspect, the disclosure features, a cell, or isolated preparation of cells, described herein.

In another aspect, the disclosure features, a high glycine optimized sequence encoding collagen 7 described herein.

In another aspect, the disclosure features an isolated preparation of cells described herein which can further comprise any of culture medium, and collagen 7, or functional fragment thereof, produce by said cell.

In another aspect, the disclosure features, a method of making a cell suitable for expressing collagen 7, or functional fragment thereof, comprising:

recombinantly manipulating a cell, e.g., a mammalian cell, e.g., a mammalian cell described herein, to express recombinant collagen 7, or functional fragment thereof; and

optionally, recombinantly manipulating said cell to express one or more polypeptides, e.g., one or more polypeptides that increase collagen 7 production in the cell (e.g., prolidase and/or prolyl hydroxylase);

thereby making a cell suitable for expressing recombinant collagen 7.

In one embodiment, the method comprises recombinantly manipulating a cell to express a collagen 7 encoded by a high glycine codon optimized nucleic acid sequence, e.g., a high glycine codon optimized nucleic acid sequence described herein.

In an embodiment of the method, the cell is recombinantly manipulated to express collagen 7, or a functional fragment thereof, and the cell is recombinantly manipulated to express one or more polypeptides, e.g., that increase the expression of collagen 7 in the cell. In one embodiment, the cell is recombinantly manipulated to express collagen 7, or a functional fragment thereof before said cell is recombinantly manipulated to express one or more polypeptides, e.g., that increase the expression of collagen 7 in the cell, e.g., one or more of prolidase, prolyl hydroxylase, glycosyl transferase, and functional fragments thereof.

In one embodiment, the cell is recombinantly manipulated to express collagen 7, or a functional fragment thereof after said cell is recombinantly manipulated to express one or more polypeptides, e.g., that increase the expression of collagen 7 in the cell, e.g., one or more of prolidase, prolyl hydroxylase, glycosyl transferase, and functional fragments thereof.

In an embodiment of the method, the cell is recombinantly manipulated to express collagen 7, or a functional fragment thereof, at the same time said cell is recombinantly manipulated to express one or more polypeptides, e.g., that increase the expression of collagen 7 in the cell, e.g., one or more of prolidase, prolyl hydroxylase, glycosyl transferase, and functional fragments thereof.

In another aspect, the invention features, collagen 7, or a functional fragment thereof, made by a method described herein.

In another aspect, the invention features, a purified or isolated preparation of collagen 7, or functional fragment thereof, made by a method described herein.

In another aspect, the invention features, a purified or isolated preparation of collagen 7, or functional fragment thereof, wherein at least 30, 40, 50, 60, 70, 80, 90 or 95% of said collagen 7, or functional fragment thereof, is incorporated into homotrimers.

In another aspect, the invention features, a purified or isolated preparation of collagen 7, or a functional fragment thereof, wherein at least 30, 40, 50, 60, 70, 80, 90 or 95% of said collagen 7, or functional fragment thereof, is incorporated into hexamers.

In another aspect, the invention features, a method of purifying collagen 7, or a functional fragment thereof, comprising:

providing conditioned cell medium, e.g., from culture of a cell described herein;

subjecting the collagen 7, or functional fragment thereof, from said medium to an anion exchange chromatography, e.g., with Q sepharose;

thereby purifying collagen 7, or a functional fragment thereof.

In an embodiment, the method comprises:

providing conditioned cell medium, e.g., from culture of a cell described herein;

optionally, precipitating protein, e.g., with ammonium sulfate, to form precipitated protein;

solubilizing the precipitated protein to form solubilized protein;

dialyzing the solubilized protein to form dialysate;

sedmenting the dialyzed sample to form a supernatant; and

subjecting the supernatant to an anion exchange chromatography, e.g., with Q sepharose;

thereby purifying collagen 7, or functional fragment thereof.

The details of one or more embodiments of the invention are set forth in the description below. Other features, objects, and advantages of the invention will be apparent from the description and the drawings, and from the claims.

### DETAILED DESCRIPTION

### Definitions

“Recombinantly manipulated to express” or “genetically manipulated to express”, as used herein, refers to a cell which has been modified so as to express a protein. Exemplary modifications include, the introduction of a nucleic acid which encodes the protein, or the placement of an endogenous sequence encoding the protein under control of a sequence other than the native endogenous sequence, e.g., by introduction of a sequence that activates an endogenous gene.

Isolated nucleic acid molecules, as used herein, means the nucleic acids have been separated from the nucleic acids of the genomic DNA or cellular RNA of their source origin. This includes nucleic acid molecules obtained by suitable methods, including, but not limited to, chemical methods, combinations of chemical and biological methods, and isolated recombinant nucleic acid molecules.

Recombinant, as used herein, in reference to a nucleic acid molecule, pertains to nucleic acid molecules which have been engineered using molecular biological techniques. Recombinant, as used herein, in reference to a protein or polypeptide molecule, pertains to a protein or polypeptide molecule expressed utilizing isolated nucleic acid molecules or recombinant nucleic acid molecules.

High glycine optimized or high glycine codon optimized, as used herein, refers to a nucleic acid sequence that encodes collagen 7, or a functional fragment thereof. The sequence includes at least one glycine codon that is other than the most common glycine codon, which is referred to herein as a less common codon. In an embodiment, the less common glycine codon is other than the most common glycine codon for the cell in which the sequence will be expressed. By way of example, if the sequence is to be expressed in CHO cells, the less common glycine codon is other than the most common glycine codon in CHO cells. In an embodiment, the less common glycine codon is a less common glycine codon for a cell referred to herein, e.g., a CHO or HEK cell. In embodiments, the sequence includes at least one, and in embodiments, at least 10, 20 or 30, less common glycine codons that is not present in the native human sequence for collagen 7. In an embodiment at least 10, 20, 30, 40, 50, 60, 70, 80, 90, or 100% of the glycine codons are less common glycine codons.

### Collagen 7

As a major component of anchoring fibrils, collagen 7 functions in maintaining tissue integrity. Anchoring fibrils are structural elements that serve as attachment complexes at the interface between the epithelial and mesenchymal layers of several tissues, including the skin, oral mucosa, and cervix (Chung et al. Dermatol Clin 28(1): 93-105 (2010)). In the skin, anchoring fibrils extend from the lower portion of the epidermal basement membrane to the underlying papillary dermis, securing the association between the epidermal basement membrane and the papillary dermis (Varki et al. J Med Genet 44:181-192 (2007)). This association aids to provide and maintain cohesion between the epidermis and dermis, contributing to the integrity to the skin, which is critical for its proper structure, function, and homeostasis (Villone et al. J Biol Chem 283(36): 24506-24513 (2008)).

Nucleic acids that encode collagen 7 can be used in the methods described herein. High glycine codon optimized sequences are particularly suitable. An exemplary high glycine codon optimized nucleotide sequence for human collagen 7 is as follows:

(SEQ ID NO: 1)
aagcttcgaagtttaaactgagtgccgccaccatgaccctgcggctgctg
gtggctgccctgtgtgctggcatcctggccgaggctcctagagtgcgggc
ccagcaccgcgagagagtgacctgcaccagactgtacgccgccgatatcg
tgttcctgctggacggctcctcctccatcggccggtccaacttccgggaa
gtgcggtccttcctggaaggcctggtgctgcctttctccggcgctgcctc
tgcccagggcgtcagattcgccaccgtgcagtactccgacgacccccgga
ccgagttcggcctggatgctctgggctctggcggcgacgtgatccgggcc
atcagagagctgtcctacaagggcggcaacacccggacaggcgccgctat
cctgcacgtggccgaccatgtgtttctgccccagctggccagacccggcg
tgcccaaagtgtgcatcctgatcaccgacggcaagtcccaggacctggtg
gacaccgccgctcagagactgaagggccagggcgtgaagctgttcgccgt
gggcatcaagaacgccgaccccgaggaactgaagcgggtggcctcccagc
ctacctccgatttcttcttcttcgtgaacgacttctccatcctgcggacc
ctgctgcccctggtgtctcggagagtgtgtaccaccgctggcggcgtgcc
agtgacccggcctcctgacgattctacctccgcccctcgggatctggtgc
tgtccgagccttccagccagtccctgagagtgcagtggaccgccgcctct
ggccccgtgaccggctacaaggtgcagtacacccctctgaccggcctggg
ccagcctctgccttctgagcggcaagaagtgaacgtcccagccggcgaga
catccgtgcggctgagaggcctgaggcccctgaccgagtaccaagtgacc
gtgatcgccctgtacgccaacagcatcggcgaggccgtgtccggcaccgc
cagaaccacagccctggaaggacccgagctgaccatccagaacaccaccg
cccactccctgctggtcgcttggagatctgtgcctggcgccaccggctat
cgcgtgacctggcgagttctgtctggcggccctacccagcagcaagagct
gggccctggacagggctctgtgctgctgagggacctggaacccggcaccg
actacgaagtgacagtgtccaccctgttcggcagatccgtgggccctgcc
acctctctgatggccagaaccgacgcctccgtggaacagaccctgaggcc
tgtgatcctgggccccaccagcatcctgctgagctggaacctggtgcccg
aggccagaggctaccggctggaatggcggagagagacaggcctggaacct
ccccagaaggtggtcctgccctccgacgtgaccagataccagctggatgg
cctgcagcctggcaccgagtacagactgaccctgtacaccctgctcgagg
gccacgaggtggccacccctgctacagtggtgcctaccggccctgagctg
cccgtgtcccctgtgaccgatctgcaggccaccgagctgcctggccagcg
cgtcagagtgtcttggtccccagtgccaggcgctacccagtaccggatca
tcgtgcggtccacacagggcgtggaaagaaccctggtgctccccggctcc
cagaccgccttcgacctggatgatgtgcaggccggcctgagctacaccgt
gcgggtgtccgctagagtgggccctagagaaggctccgccagcgtgctga
ccgtgcgcagagagcctgaaacccctctggccgtgcccggactgcgggtg
gtggtgtctgatgccaccagagtcagagtggcctggggccctgtgccagg
ggcctccggcttcagaatctcctggtccaccggctctggccctgagtcct
ctcagaccctgccccctgactccaccgccaccgatatcaccggactgcag
cccggaaccacctaccaggtggccgtgtctgtgctgaggggcagagaaga
gggcccagccgccgtgatcgtggccaggacagatcctctgggcccagtgc
ggaccgtgcacgtgacccaggccagctccagctccgtgaccatcacctgg
accagagtccctggcgctacaggctacagagtgtcctggcactctgccca
cggccccgagaagtcccagctggtgtctggcgaggccaccgtggctgaac
tggacggcctcgagcccgacacagagtacacagtgcacgtgcgcgcccat
gtggctggcgtggacggacctcctgcttccgtggtcgtgcgcaccgctcc
tgagcccgtgggaagagtgtcccggctgcagatcctgaacgcctccagcg
acgtgctgcggatcacctgggtcggagtgaccggcgctaccgcttacaga
ctggcttggggcagatctgagggcggacccatgcggcatcagatcctgcc
tggcaacaccgactccgccgagatcagaggactggaaggcggcgtgtcct
actctgtgcgcgtgaccgccctcgtgggcgacagagaaggcacccccgtg
tccatcgtggtcaccacccctccagaggcccctccagctctgggcaccct
gcatgtggtgcagcggggcgagcactccctgagactgagatgggagcctg
tgcctcgggcccagggcttcctgctgcattggcagcctgaaggcggccaa
gagcagtctagggtgctgggccccgagctgtccagctaccacctggacgg
actggaaccagccacccagtacagagtgcggctgtccgtgctgggacctg
ccggcgagggaccttctgccgaagtgaccgccaggaccgagtcccctcgg
gtgccctccatcgagctgagagtggtggataccagcatcgacagcgtgac
cctggcctggacccctgtgtcccgggcctcttcctacatcctgtcttgga
ggcctctgaggggcccaggccaagaggtgccaggctcccctcagacactg
ccaggcatcagctcctcccagcgcgtgacaggactcgagcctggggtgtc
ctacatcttctccctgacccccgtcctggacggcgtgcgcggacctgagg
cttctgtgacccagaccccagtgtgccccagaggcctggccgacgtggtc
tttctgcctcacgccacccaggacaacgcccacagagccgaggctaccag
acgggtgctcgagagactggtgctggccctgggaccactgggcccacagg
ctgtgcaagtgggcctgctgtcttactcccaccggccctcccccctgttc
cccctgaacggctctcacgacctgggcatcatcctgcagcggatccggga
catgccctacatggacccctccggcaacaacctgggcaccgccgtggtca
cagcccaccggtacatgctggcccccgatgctcctggcagacggcagcat
gtccccggcgtgatggtgctgctcgtggacgagcccctgcggggcgacat
cttcagccctatcagagaggcccaggctagcggcctgaacgtggtcatgc
tgggcatggctggcgccgaccctgagcagctgagaaggctggcccctggc
atggactccgtgcagaccttctttgccgtggacgacggccccagcctgga
tcaggctgtgtctggcctggctaccgccctgtgtcaggcctccttcacca
cccagcccagacccgagccttgccccgtgtactgccctaagggccagaag
ggcgagcccggcgagatgggcctgagaggacaagtgggacctccaggcga
tcccggcctgcctggaagaacaggcgctcctggacctcagggccctcctg
gctctgctaccgctaagggcgagagaggcttcccaggcgccgacggcaga
cctggctctccaggcagagccggcaatcctggaacacctggcgccccagg
cctgaagggatctcctggcttgcctggccctaggggcgaccctggcgaaa
gaggacctagaggccctaaaggcgagccaggcgcccctggccaagtgatc
ggcggagaaggacctggactgcccggcagaaagggcgatcctggcccttc
tggcccacccggcccaagaggtcctctgggagatcctggaccaaggggcc
caccaggcctgcccggaacagctatgaagggcgataagggcgacaggggc
gagcggggaccaccaggaccaggcgaaggtggaatcgctcccggcgaacc
tgggctgccaggactgcctggatctcccggaccacagggacctgtgggcc
cacctggcaagaagggggagaaaggcgactccgaggacggggctccagga
ttgcccggccaaccaggctctcctggcgaacagggtcccagaggacctcc
cggcgctatcggcccaaagggggacagaggattccctggcccactgggcg
aggctggcgaaaaaggcgaacgcggaccccctggccctgccggcagtaga
ggacttcctggcgttgccggcagaccaggcgccaagggacctgaaggccc
tccaggccctaccggaaggcagggcgaaaagggggaacctggcaggccag
gcgacccagctgttgtgggaccagccgtggctggacccaaaggcgagaaa
ggggatgtgggacccgctgggcctagaggcgctactggcgttcaggggga
aagaggcccccctggactcgtgctgcctggggatccaggtcctaaggggg
atcccggcgatagaggcccaatcggcctgaccggcagagctggtccccct
ggcgattccggtcctcccggggaaaaaggggaccccggtagaccaggtcc
cccaggccctgttggccctcgcggaagagatggcgaagtgggagaaaagg
gcgacgaaggacccccaggggacccaggacttccaggcaaggctggggag
agaggactgaggggcgctccaggtgtcagaggccctgtcggcgagaaggg
ggatcagggcgatccaggcgaggacggcagaaacggctcccctggctcta
gtggtccaaaaggcgaccggggagagcctgggcctcctgggccaccaggc
agactggtcgataccggacctggggccagagagaagggcgaaccagggga
taggggccaagaaggcccacgaggaccaaagggcgacccaggattgcctg
gcgctcctggcgagaggggcatcgagggctttagaggtccacccggtccc
caaggcgaccccggcgttaggggacctgctggggagaagggcgacagagg
cccacccggactggacggcagatctggcctggatggcaagcctggcgccg
ctggcccatctggacctaacggcgctgctggcaaagccggggaccctgga
cgagatggactgccagggctgcggggagaacagggccttccaggaccttc
aggaccacctggcctccctggcaagcccggggaggatggaaagcccggcc
tgaatggaaaaaacggggaacccggggatcctggggaggacggacgcaag
ggggaaaagggcgattccggcgcctctggcagagagggcagggacggacc
aaaaggggagcgcggagcacccggcattctgggtcctcaggggccacctg
gattgccaggtccagttggtcctcctggccaggggtttcccggcgtccca
ggcggtacagggcctaaaggggatagaggcgagacaggcagcaaagggga
acaggggctcccaggcgaaaggggcttgagaggcgagcctggctccgtgc
ctaacgtggacagactgctggaaaccgccggcatcaaggcctccgccctg
cgcgagatcgtggaaacctgggacgagtcctccggctccttcctgcccgt
gcctgagcgcagaaggggcccgaaaggggactctggcgagcaaggaccac
ccggcaaagagggacccatcggcttccctggggagcgggggttgaaaggc
gataggggagatccaggcccacaagggcctccagggctggcacttggaga
gcgtggtcctccaggaccaagcggactggcaggggagcccggaaagcctg
gaatccccgggttgcctggtagagccggcggagtgggcgaagcaggcagg
cctggggaacgcggagagagaggcgaaaagggcgaaagaggggagcaggg
ccgcgacggtccccccggactacctggaactccagggcccccaggacccc
ccggacctaaggtgtccgtggatgagcctggccccggactgagcggagaa
caaggtccacctggcttgaagggtgccaagggggagccaggctctaacgg
cgatcaagggcccaagggggatcggggagtgcctggcatcaaaggggacc
ggggcgaacccggtcctagagggcaagacggaaaccccggcttgccgggc
gaacggggaatggctggtcccgagggaaagccaggcttgcagggacctag
ggggcctcccggtcctgtgggtggacatggcgatccgggtccaccaggtg
ctccaggactcgctggtccagcaggccctcagggaccatccggcctgaaa
ggggaaccaggcgaaactggccccccaggcagaggcctgacaggccctac
tggtgctgtgggcctccctggacctcctggccctagtggactcgtgggcc
ctcagggctctcccggactgccaggccaagtgggcgagactggaaaaccc
ggggctcccggcagggatggcgcttctggaaaagacggcgataggggcag
ccctggcgtgcccggtagtccagggctacctggccctgtgggtcccaaag
gggagcctggacctacaggcgcaccaggccaggctgtagtggggctgcct
ggcgctaaaggcgagaagggtgctcctggcggcctggctggcgatctcgt
tggagaacctggcgccaagggcgaccgtggcttgccaggacctcgcggcg
agaaaggcgaagctggcagagctggcgagcctggggacccaggcgaagat
ggccagaaaggcgctcccggccctaagggattcaagggcgatccgggcgt
gggcgtgccaggctctccaggtcctcctggaccacccggtgtcaagggcg
atttgggccttcctggcctgccaggggcacctggcgtcgtgggctttcct
ggacagaccggcccacggggagagatgggacagccaggccccagcggaga
aagagggctggctggcccgcctggcagggaaggcataccaggcccattgg
ggcctccaggcccacctggatctgtggggcctcctggcgcctctggactg
aaaggcgacaaaggcgatcctggtgtcggcctgccaggcccaagaggcga
gaggggagagcccggcatcaggggcgaagatggacggcctggccaagagg
gccctcggggattgaccggccctcctggatccagaggcgaacggggggag
aagggggacgtgggctctgctggcctcaaaggcgacaagggggactccgc
cgtgattctgggccctcccggacctcggggagctaagggggacatgggag
agaggggtccacggggactggatggggacaagggaccacgcggagacaac
ggcgacccgggggataagggctccaagggcgaacctggcgataagggatc
cgctggactgcctggcctgaggggcctgctgggacctcaaggacaaccag
gcgccgcaggcatccctggcgaccctggatctcctggaaaggacggcgtg
cccggcatccgcggagaaaagggggatgtcggcttcatgggccccagggg
gctgaagggggaaaggggagtgaagggcgcttgcggcctcgatggggaaa
agggggacaagggggaggctggccctccaggacgacctggactggctggc
cacaagggcgaaatgggagagccaggcgtgcccggacagtccggcgctcc
aggcaaagagggcctgatcggccccaaaggcgatagaggatttgacggcc
agcctggcccaaagggcgatcaaggcgaaaaaggggagagaggcaccccc
ggcatcggcggctttccaggcccctctggaaacgatggctctgccggccc
acctgggccacctggtagtgtgggaccaagaggccccgagggactgcagg
gacagaaaggcgagagagggccccctggcgagagagttgtgggagcacct
ggcgttcccggcgcacccggcgaaaggggagaacaaggcagacctggacc
agccggaccccgtggggaaaaaggcgaggccgccctgaccgaggacgaca
tcagaggcttcgtgcggcaagagatgtcccagcactgcgcctgtcagggc
cagtttatcgcctccggcagcagacccctgccttcctacgctgccgatac
cgccggctctcagctgcacgctgtgcctgtgctccgggtgtcccacgccg
aggaagaggaaagagtccctcctgaggacgacgagtacagcgagtactct
gagtattccgtggaagagtaccaggatcccgaggccccttgggacagcga
cgacccttgctccctgcctctggatgagggctcctgcaccgcctacaccc
tgagatggtatcaccgggccgtgacaggctccaccgaggcctgtcaccct
ttcgtgtatggcggctgcggcggcaacgccaatagattcggcacccgcga
ggcctgcgagcggagatgtcctcccagagtggtgcagtcccagggcaccg
gcacagcccaggactgatagtctagagtggccggcc

An amino acid sequence for human collagen 7 is as follows:

(SEQ ID NO: 2)
mtlrllvaalcagilaeaprvraqhrervtctrlyaadivflldgsssig
rsnfrevrsfleglvlpfsgaasaqgvrfatvqysddprtefgldalgsg
gdvirairelsykggntrtgaailhvadhvflpqlarpgvpkvcilitdg
ksqdlvdtaaqrlkgqgvklfavgiknadpeelkrvasqptsdffffvn
dfsilrtllplvsrrvcttaggvpvtrppddstsaprdlvlsepssqslr
vqwtaasgpvtgykvqytpltglgqplpserqevnvpagetsvrlrglrp
lteyqvtvialyansigeaysgtarttalegpeltiqnttahsllvawrs
vpgatgyrytwrvlsggptqqqelgpgqgsvllrdlepgtdyevtvstlf
grsvgpatslmartdasveqtlrpvilgptsillswnlypeargyrlewr
retgleppqkvvlpsdvtryqldglqpgteyrltlytlleghevatpatv
vptgpelpvspvtdlqatelpgqrvrvswspvpgatqyriivrstqgver
tlvlpgsqtafdlddvqaglsytvrvsarvgpregsasvltvrrepetpl
avpglrvvvsdatrvrvawgpvpgasgfriswstgsgpessqtlppdsta
tditglqpgttyqvaysvlrgreegpaavivartdplgpvrtvhvtqass
ssvtitwtrvpgatgyrvswhsahgpeksqlvsgeatvaeldglepdtey
tvhvrahvagvdgppasvvvrtapepvgrvsrlqilnassdvlritwvgv
tgatayrlawgrseggpmrhqilpgntdsaeirgleggvsysvrvtalvg
dregtpvsivvttppeappalgtlhvvqrgehslrlrwepvpraqgfllh
wqpeggqeqsrvlgpelssyhldglepatqyrvrlsvlgpagegpsaevt
artesprvpsielrvvdtsidsvtlawtpvsrassyilswrplrgpgqev
pgspqtlpgisssqrvtglepgvsyifsltpvldgvrgpeasvtqtpvcp
rgladvvflphatqdnahraeatrrvlerlvlalgplgpqavqvgllsys
hrpsplfplngshdlgiilqrirdmpymdpsgnnlgtavvtahrymlapd
apgrrqhvpgvmvllvdeplrgdifspireaqasglnvvmlgmagadpeq
lrrlapgmdsvqtffavddgpsldqaysglatalcqaslttqprpepcpv
ycpkgqkgepgemglrgqvgppgdpglpgrtgapgpqgppgsatakgerg
fpgadgrpgspgragnpgtpgapglkgspglpgprgdpgergprgpkgep
gapgqviggegpglpgrkgdpgpsgppgprgplgdpgprgppglpgtamk
gdkgdrgergppgpgeggiapgepglpglpgspgpqgpvgppgkkgekgd
sedgapglpgqpgspgeqgprgppgaigpkgdrgfpgplgeagekgergp
pgpagsrglpgvagrpgakgpegppgptgrqgekgepgrpgdpavvgpav
agpkgekgdvgpagprgatgvqgergppglvlpgdpgpkgdpgdrgpigl
tgragppgdsgppgekgdpgrpgppgpvgprgrdgevgekgdegppgdpg
lpgkagerglrgapgvrgpvgekgdqgdpgedgrngspgssgpkgdrgep
gppgppgrlvdtgpgarekgepgdrgqegprgpkgdpglpgapgergieg
frgppgpqgdpgvrgpagekgdrgppgldgrsgldgkpgaagpsgpngaa
gkagdpgrdglpglrgeqglpgpsgppglpgkpgedgkpglngkngepgd
pgedgrkgekgdsgasgregrdgpkgergapgilgpqgppglpgpvgppg
qgfpgvpggtgpkgdrgetgskgeqglpgerglrgepgsvpnvdrlleta
gikasalreivetwdessgsflpyperrrgpkgdsgeqgppgkegpigfp
gerglkgdrgdpgpqgppglalgergppgpsglagepgkpgipglpgrag
gvgeagrpgergergekgergeqgrdgppglpgtpgppgppgpkvsvdep
gpglsgeqgppglkgakgepgsngdqgpkgdrgvpgikgdrgepgprgqd
gnpglpgergmagpegkpglqgprgppgpvgghgdpgppgapglagpagp
qgpsglkgepgetgppgrgltgptgavglpgppgpsglvgpqgspglpgq
vgetgkpgapgrdgasgkdgdrgspgvpgspglpgpvgpkgepgptgapg
qavvglpgakgekgapgglagdlvgepgakgdrglpgprgekgeagrage
pgdpgedgqkgapgpkgfkgdpgvgvpgspgppgppgvkgdlglpglpga
pgvvgfpgqtgprgemgqpgpsgerglagppgregipgplgppgppgsvg
ppgasglkgdkgdpgvglpgprgergepgirgedgrpgqegprgltgppg
srgergekgdvgsaglkgdkgdsavilgppgprgakgdnngergprgldg
dkgprgdngdpgdkgskgepgdkgsaglpglrgllgpqgqpgaagipgdp
gspgkdgvpgirgekgdvgfmgprglkgergvkgacgldgekgdkgeagp
pgrpglaghkgemgepgvpgqsgapgkegligpkgdrgfdgqpgpkgdqg
ekgergtpgiggfpgpsgndgsagppgppgsvgprgpeglqgqkgergpp
gervvgapgvpgapgergeqgrpgpagprgekgeaalteddirglvrqem
sqhcacqgqfiasgsrplpsyaadtagsqlhavpvlrvshaeeeervppe
ddeyseyseysveeyqdpeapwdsddpcslpldegsctaytlrwyhravt
gsteachpfvyggcggnanrfgtreacerrcpprvvqsqgtgtaqd

### Prolidase

Prolidase is a cytosolic imidodipeptidase, which specifically splits imidodipeptides with C-terminal proline or hydroxyproline residues. The enzyme plays an important role in the recycling of proline from imidodipeptides, mostly derived from degradation products of collagen, for resynthesis of collagen and other proline containing proteins. Specific host cells may require supplementation of prolidase to ensure proper synthesis of the recombinant collagen protein (as referenced in (Miltyk et al. J Biochem 144(3): 409-414 (2008)). Host cells described herein, recombinantly manipulated to express collagen 7, may be recombinantly manipulated to also express human prolidase. An exemplary amino acid sequence for human prolidase is as follows:

(SEQ ID NO: 4)
maaatgpsfwignetikvplalfalnrqrlcerlrknpavqagsivvlqg
geetqryctdtgvlfrqesffhwafgvtepgcygvidvdtgkstlfvprl
pashatwmgkihskehfkekyavddvqdeiasvitsqkpsviltlrgvnt
dsgsvcreasfdgiskfevnntilhpeivecrviktdmelevlrytnkis
seahrevmkavkvgmkeyeleslfehycysrggmrhssytcicgsgensa
vlhgagapndrtiqngmclfdmggeyycfasditcsfpangkftadqkav
yeavlrssravmgamkpgvwwpdmhrladrihleelahmgilsgsvdamv
qahlgavfmphglghflgidvhdvggypgvridepglrslrtarhlqpgm
vitvepgiyfidhlldealadparasflnrevlqrfrgfggvrieedvvv
tdsgielitcvprtveeieacmagcdkaftpfsgpk

An exemplary nucleic acid sequence encoding human prolidase is provided below:

| (SEQ ID NO: 3) | |
|---|---|
| 1 | ccggtgccgg gcgaacatgg cggcggccac cggaccctcg ttttggctgg ggaatgaaac |
| 61 | cctgaaggtg ccgctggcgc tctttgcctt gaaccggcag cgcctgtgtg agcggctgcg |
| 121 | gaagaaccct gctgtgcagg ccggctccat cgtggtcctg cagggcgggg aggagactca |
| 181 | gcgctactgc accgacaccg gggtcctctt cctccaggag tccttctttc actgggcgtt |
| 241 | cggtgtcact gagccaggct gctatggtgt catcgatgtt gacactggga agtcgaccct |
| 301 | gtttgtgccc aggcttcctg ccagccatgc cacctggatg ggaaagatcc attccaagga |
| 361 | gcacttcaag gagaagtatg ccgtggacga cgtccagtac gtagatgaga ttgccagcgt |
| 421 | cctgacgtca cagaagccct ctgtcctcct cactttgcgt ggcgtcaaca cggacagcgg |
| 481 | cagtgtctgc agggaggcct cctttgacgg catcagcaag ttcgaagtca acaataccat |
| 541 | tcttcaccca gagatcgttg agagccgagt gtttaagacg gatatggagc tggaggttct |
| 601 | gcgctatacc aataaaatct ccagcgaggc ccaccgtgag gtaatgaagg ctgtaaaagt |
| 661 | gggaatgaaa gaatatgggt tggaaagcct cttcgagcac tactgctact cccggggcgg |
| 721 | catgcgccac agctcctaca cctgcatctg cggcagtggt gagaactcag ccgtgctaca |
| 781 | ctacggacac gccggagctc ccaacgaccg aacgatccag aatggggata tgtgcctgtt |
| 841 | cgacatgggc ggtgagtatt actctgtcgc ttccgacatc acctgctcct ttccccgcaa |
| 901 | cggcaagttc actgcagacc agaaggccgt ctatgaggca gtgctgctga gctcccgtgc |
| 961 | cgtcatgggt gccatgaagc caggtgactg gtggcctgac atcgaccgcc tggctgaccg |
| 1021 | catccacctg gaggagctgg cccacatggg catcctgagc ggcagcgtgg acgccatggt |
| 1081 | ccaggctcac ctgggggccg tgtttatgcc tcacgggctt ggccacttcc tgggcattga |
| 1141 | cgtgcacgac gtgggaggct acccagaggg cgtggagcgc atcgacgagc ccggcctgcg |
| 1201 | gagcctgcgc actgcacggc acctgcagcc aggcatggtg ctcaccgtgg agccgggcat |
| 1261 | ctacttcatc gaccacctcc tggatgaggc cctggcggac ccggcccgcg cctccttcct |
| 1321 | taaccgcgag gtcctgcagc gctttcgcgg ttttggcggg gtccgcatcg aggaggacgt |
| 1381 | cgtggtgatc gacagcggca tagagctgct gacctgcgtg ccccgcactg tggaagagat |
| 1441 | tgaagcatgc atggcaggct gtgacaaggc ctttaccccc ttctctggcc ccaagtagag |
| 1501 | ccagccagaa atcccagcgc acctgggggc ctggccttgc aacctctttt cgtgatgggc |
| 1561 | agcctgctgg tcagcactcc agtagcgaga gacggcaccc agaatcagat cccagcttcg |
| 1621 | gcatttgatc agaccaaaca gtgctgtttc ccggggagga aacacttttt taattaccct |
| 1681 | tttgcaggca ccacctttaa tctgttttat accttgctta ttaaatgagc gacttaaaat |
| 1741 | gattgaaaat aatgctgtcc tttagtagca agtaaaatgt gtcttgctgt catttatatt |
| 1801 | ccttttccca ggaaagaagc atttctgata ctttctgtca aaaatcaata tgcagaatgg |
| 1861 | catttgcaat aaaaggtttc ctaaaatg |

### Glycosyl Transferase

Mammalian host cells, such as CHO cells, may be employed to produce glycosylated recombinant proteins, such as collagen 7, because they are equipped with glycosylation machinery similar to the human. However, a notable difference concerns sialylation: N-linked glycans of human origin carry terminal sialic acid residues in both K2,3- and K2,6-linkages, whereas only K2,3 terminal sialic acids are found in glycoproteins from CHO and BHK cells. Indeed, these cell lines lack a functional copy of the gene encoding K2,6-sialyltransferase (Bragonzi et al. Biochim Biophys Acta 1474(3): 273-82 (2000)). Host cells may be recombinantly manipulated to express the human glycosyl aminotransferase, rST6Gal1, before, after, or simultaneously with manipulating said host cell to recombinantly express collagen 7 or collagen 7 and prolidase.

Amino acid sequence for rattus norvegicus ST6 beta-galactosamide alpha-2,6-sialyltransferase 1 (St6gal1), transcript variant 1 (rST6Gal1)

(SEQ ID NO: 5)
mihtnlkkkfslfilvfllfavicvwkkgsdyealtlqakefqmpksqek
vamgsasqvvfsnskqdpkedipilsyhrvtakvkpqpsfqvwdkdstys
klnprilkiwrnylnmnkykvsykgpgvkfsvealrchlrdhvnvsmiea
tdfpfnttewegylpkenfrtkvgpwqrcavvssagslknsqlgreidnh
davlrfngaptdnfqqdvgskttirlmnsqlvttekrflkdslytegili
vwdsyhadipkwyqkpdynffetyksyrrinpsqpfyilkpqmpwelwdi
iqeisadliqpnppssgmlgiiimmticdqvdiyeflpskrktdvcyyhq
kffdsactmgayhpllfeknmvkhlnegtedylfgkatlsgfrnirc

Nucleotide sequence for human ST6 beta-galactosamide alpha-2,6-sialyltransferase 1 (St6gal1), transcript variant 1 (rST6Gal1) can be optimized.

### Prolyl Hydroxylase

An exemplary prolyl hydroxylase is described below:

| (SEQ ID N: X) | |
|---|---|
| 1 | mahhhhhhlp alklaleyiv pcmnkhgicv vddflgketg qqigdevral hdtgkftdgq |
| 61 | lvsqksdssk dirgdkitwi egkepgceti gllmssmddl irhcngklgs ykingrtkam |
| 121 | vacypgngtg yvrhvdnpng dgrovtciyy lnkdwdakvs ggilrifpeg kagfadiepk |
| 181 | fdrllffwsd rrnphevqpa yatryaitvw yfdaderara kvkyltgekg vrvelnkpsd |
| 241 | svgkdvf |

### Heat Shock Protein 47 (HSP47)

HSP47 is a chaperone protein resident in the endoplasmic reticulum which functions in procollagen formation. HSP47 assists in the translocation of procollagen into the endoplasmic reticulum. HSP47 also helps maintain the emerging polypeptide in an unfolded state until synthesis is complete, and release of procollagen from HSP47 is driven by procollagen helical formation. Host cells of the present invention may be recombinantly manipulated to express the human HSP47, before, after, or simultaneously with manipulating said host cell to recombinantly express collagen 7 or collagen 7 and prolidase.

### Cyclophilin B (Cyp B)

Cyclophilin B is a peptidyl-prolyl cis-trans isomerase found in the endoplasmic reticulum. Cyclophilin B functions in consort with HSP47 to facilitate the folding and transport of procollagen. Host cells of the present invention may be recombinantly manipulated to express the human cyclophilin B, before, after, or simultaneously with manipulating said host cell to recombinantly express collagen 7 or collagen 7 and prolidase.

### Protein Disulfide Isomerase (PDI)

Protein Disulfide Isomerase (PDI) is an ER resident thiol oxidoreductase protein. PDI assists in protein folding in part through catalyzing of the formation, reduction, and isomerization of disulphide bonds. PDI facilitates the stabilization of collagen trimers through catalyzing the formation of interchain disulphide bonds between the C-propeptide domains. Host cells of the current invention may be recombinantly manipulated to express the human PDI, before, after, or simultaneously with manipulating said host cell to recombinantly express collagen 7 or collagen 7 and prolidase.

### Oxoglutarate Carrier (OGC)

Oxoglutarate carrier (OGC) is a mitochondrial resident protein which transports the α-ketoglutarate across the inner membrane of the mitochondria and facilitates the coupling of decarboxylated α-ketoglutarate to proline. Host cells may be recombinantly manipulated to express the human OGC, before, after, or simultaneously with manipulating said host cell to recombinantly express collagen 7 or collagen 7 and prolidase.

### Vectors

Suitable vectors for use herein are those that can express collagen 7, prolidase, glycosyl-transferase, HSP47, cyclophilin B, PDI, OGC, or a molecular chaperone involved in procollagen assembly or folding, or a functional portion thereof. In order to express the proteins described herein, the nucleotide sequence encoding the appropriate protein, or a functional equivalent, can be inserted into a suitable vector. A suitable vector contains the necessary and appropriate transcriptional and translational control sequences for expression of the inserted nucleic acid sequence. Standard methods, known to those skilled in the art, may be used to construct the recombinant expression vectors containing the nucleic acid sequences described herein. These methods include, but not limited to, in vitro recombinant techniques, synthetic techniques, and in vivo recombination/genetic recombination; the choice of method depends on the nature of the specific nucleotide fragments and may be determined by persons skilled in the art.

Suitable vectors for use herein may contain an origin of replication and a restriction endonuclease sequence site. Persons skilled in the art would have knowledge of suitable origin of replication and restriction endonuclease sequences for use in the host cell. Suitable vectors for use herein may contain sequence elements to aid transcription, including, but not limited to, promoter and enhancer elements. Persons skilled in the art would have knowledge of various transcriptional control elements, including but not limited to, promoters, inducible promoters, and enhancer elements, that would be suitable in the host cell. Suitable vectors for use herein may also contain a selectable marker gene that encodes a product necessary for the host cell to grow and survive under specific conditions, aiding in the selection of host cells into which the vector has been introduced. Typical selection genes may include, but not limited to, genes encoding a protein that confers resistance to an antibiotic, drug, or toxin (e.g. tetracycline, ampicilin, neomycin, hygromycin, etc). Persons skilled in the art would have knowledge of coding sequences for suitable selectable markers and reporter genes for use in the host cell.

Expression vectors described herein can be introduced into host cells via conventional transformation or transfection techniques. Transformation and transfection techniques include, but not limited to, calcium phosphate or calcium chloride coprecipitation, DEAE-dextran-mediated transfection, lipofectamine, electroporation, microinjection, and viral mediated transfection (as referenced in U.S. Pat. No. 6,632,637 (McGrew)). Persons skilled in the art would have knowledge of suitable transformation and transfection methods based on the host cell/vector combination. For long term, high yield production of recombinant proteins, stable expression of the recombinant protein may be preferred. Host cells that stably express the recombinant protein may be engineered.

### Cells

The recombinant expression vectors described herein may be introduced into a suitable host cell, which may include a living cell capable of expressing the protein coding region from the defined recombinant expression vector. The term “host cell” refers not only to the particular subject cell but to the progeny or potential progeny of the particular subject cell. Because certain modifications may occur in succeeding generations due to either mutation or environmental influences, such progeny may not in fact, be identical to the parent cell, but are still included within the scope of the term as used herein. Various host cell expression systems may be utilized to express the nucleic acid molecules described herein. These include, but are not limited to yeast or fungi, transformed with recombinant yeast or fungi expression vectors containing the appropriate nucleic acid sequence; insect cell systems infected with recombinant virus expression vectors or transformed with recombinant plasmid expression vectors containing the appropriate nucleic acid sequence; or mammalian cell systems (e.g. primate cell, human cell, rodent cell, etc) transfected with expression vectors containing the appropriate nucleic acid sequence. Suitable host cells may include primary or transformed cell lines, including, but not limited to, fibroblasts, CHO, HEK293, C127, VERO, BHK, HeLa, COS, MDCK, etc (as referenced in U.S. Pat. No. 6,632,637 (McGrew)). Other suitable host cells are known to those skilled in the art.

Modifications, including, but not limited to, glycosylation, phosphyorylation and processing of protein products may be important to the function of a protein. Different host cells have various characteristics and mechanisms for post-translational processing and modification of proteins. A host cell that is capable of modulating expression of the nucleic acid sequences contained in the vector, or modulating expression of the vector nucleic acid sequences, or modifying and processing the gene product encoded in the vector sequence in a specific manner may be chosen. Mammalian host cells may be chosen to ensure the correct modification and processing of the recombinant protein. Such mammalian host cells may include, but not limited to, CHO, HEK293, human fibroblasts, and human keratinocytes.

### Cell Culture

Standard cell culture procedures and conditions may be used for culture of host cells described herein and are known to those skilled in the art. Host cells cultured for expression of recombinant collagen 7, such as HEK293 cells, may be cultured in routinely used cell culture media (e.g. Dulbecco's modified Eagle's medium (DMEM)/Ham's F-12 (1:1) with suitable supplementation of serum, antibiotics, etc, dependent on the application) as referenced in, ((Chen et al. J Bio Chem 277(18): 2118-2124 (2002)), (Chen et al. J Bio Chem 275: 32(11): 24429-24435 (2000)), (Chen et al. J Bio Chem 276(24): 21649-21655 (2001)).

Host cells may be engineered to express other proteins to optimize production of the recombinant collagen 7, or a functional fragment thereof. This may include, but not limited to, the coexpression of the processing enzymes prolidase and/or glycosyl-transferase described herein, by exogenously introducing isolated nucleic acid or recombinant expression vectors encoding the appropriate nucleic acid sequence, in host cells comprising collagen 7 nucleic acid sequence or recombinant expression vector. The triple helical assembly of collagen 7 often requires hydroxylation and the presence of ascorbic acid in the host cell growth media. As demonstrated in the reference, (Chen et al. J Bio Chem 277 (18): 2118-2124 (2002)), recombinant type 7 collagen produced, recovered, and purified from HEK293 cells in the presence of ascorbic acid was secreted as an approximately 900-kDa protein, corresponding to the association of three type 7 collagen monomers (each monomer 290-kDa). Ascorbic acid may be used in the host cell culture conditions to aid in proper processing of the recombinant protein. Additional supplements to the cell culture media may be added in order to aid in proper processing of the recombinant protein, including but not limited to, phospho-ascorbate (PAA), 4 mM α-ketoglutarate, FeSO₄, or Optiferrin.

### Homologous Sequences

The methods and compositions of the present invention encompass polypeptides and nucleic acids having the sequences specified, or sequences substantially identical or similar thereto, e.g., sequences at least 70%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to SEQ ID NO:2, SEQ ID NO:4 or SEQ ID NO:6 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 70&, 85%, 90%. 91%, 92%, 93%, 94%, 95%, 96%, 97%, 98% or 99% identity to SEQ ID NO:1, 3, or 5 are termed substantially identical.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to BMP-10/BMP-10 receptor nucleic acid (SEQ ID NO:1) molecules of the invention. BLAST protein searches can be performed with the XBLAST program, score=50, wordlength=3 to obtain amino acid sequences homologous to BMP-10/BMP-10 receptor (SEQ ID NO:1) protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Also included herein are sequences that hybridize under low, medium or high stringency to a recited nucleic acid. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

### Purification of Collagen 7, or a Functional Fragment Thereof

Proteins produced by recombinant methods described herein may be recovered from the host cell culture system according to standard protocols known in the art (e.g., precipitation, centrifugation, etc). Recombinant collagen 7 described herein may be secreted into the host cell medium and recovered by ammonium sulfate precipitation and subsequent centrifugation; as demonstrated in the following reference, (Chen et al. J Bio Chem 277(18): 2118-2124 (2002)). Proteins produced and recovered by recombinant and molecular biology methods described herein, may be purified according to standard protocols known in the art (e.g., dialysis, ion exchange chromatography, affinity chromatography, SDS gel electrophoresis, etc). The recombinant collagen 7 described herein may be purified to homogeneity by ion exchange chromatography; as demonstrated in the following reference, (Chen et al. J Bio Chem 277(18): 2118-2124 (2002)).

### EXEMPLIFICATION

### Example 1. Production and Purification of Collagen 7

### Subculturing and Freezing the Cells

1. Wash the cells with PBS (10 ml for P150 plate).
2. Add 6 ml of trypsin (0.05% trypsin-EDTA in PBS) and incubate at 37° C. incubator for 4 to 6 minutes. Cells will detach as a layer.
3. Add 6 ml of growth medium and then spin down at conical centrifuge at 2K for 5 minutes.
4. Resuspend the cells in growth medium and subculture cells at 1:5 ratio.
5. For freezing the cells, we use growth medium with 10% DMSO. A confluent P150 plates will give rise to approximately 20 millions of cells.

### Defrost and Re-Growth the Cells

1. Take a viral of RDEB/FB/C7 (5×10⁶) and thaw in 37° C. water bath briefly.
2. Put into a P150 plate containing 20 ml of growth medium and incubate overnight.
3. Change to the fresh medium at second day. Cells should reach confluent after 2 to 3 days.
4. Take out 30 μl medium directly and subject to immunoblot with anti-type VII collagen antibody to insure the presence of type VII collagen in the medium.

### Growth and Harvest Medium

Growth medium: DMEM/F12 (1:1) with L-glutamine and L sodium bicarbonate (Mediatech, Inc., DMEM prepares 10 L at 13.48 g/L Cat. No. 50-003-PB and Ham's F-12 Medium prepares 10 L at 10.64 g/L Cat. No. 50-040-PB), 10% FBS (Omega Scientific Cat# FB-02) and 200 μg/ml ascorbic acid (Sigma CAT# A4544) (1 ml stock solution of 100 mg/ml added to 500 ml medium).
Serum free medium: DMEM/F12 without serum and ascorbic acid.
1. Grow gene-corrected RDEB fibroblasts in P150 plates in 20 ml of growth medium till confluence.
2. Add 15 ml of serum free medium in the morning (for example Monday morning).
3. Harvest medium in the next afternoon and add back 20 ml of growth medium to the cells (Tuesday afternoon).
4. Two days later, add serum free medium in the morning again (Thursday morning).
5. Harvest the medium again next afternoon (Friday afternoon).
6. Repeat this cycle on following Monday for at least 3 to 4 months till cells detach (some time cells can go on for 6 months and still secrete a lot of type VII collagen).
The serum free media of gene corrected fibroblasts contain approximately 4 to 8 mg/L type VII collagen. After purification, between 0.7 to 1 mg of type VII collagen in normally obtained from 1 liter media.

### Purification of Type VII Collagen

### Materials:

Ammonium Sulfate
EDTA: 500 mM, pH 8
NEM: 100 mM
PMSF: 100 mM
Q Sepharose™ Fast Flow (GE Healthcare CAT#17-0510-01)

### 1× Buffer A: 65 mM NaCl

25 mM Tris-HCl pH 8.6
1 mM EDTA
   For 2 L 10× buffer A: 76.11 g NaCl
250 ml 2M Tris-HCl pH 8.6
40 ml 0.5M EDTA

### Buffer B: 50 mM Tris pH 7.8

150 mM NaCl
5 mM EDTA
2 mM MEM
2 mM PMSF

### Buffer C: 50 mM Tris pH 7.5

2 M Urea
0.5 M NaCl
1 mM EDTA
2 mM MEM
2 mM PMSF

Day 1
1. Collect conditioned cell medium and spin at 3000 rpm for 10 min in 4° C. to remove the cell debris.
2. Measure the harvest volume (Total Media collected)
3. Add inhibitors: 5 mM EDTA (100 fold), 50 μM NEM and 50 μM PMSF (2000 fold)
4. Slowly add Ammonium Sulfate powder on ice: 0.3 g/mL
5. Stir overnight at 4° C.

Day 2
6. Spin at 13,000 rpm in Beckman J2-M1 rotor 14 for 1.5-2 hours
7. Discard supernatant, then air-dry pellet for 10-15 min.
8. Bring pellet up in Buffer A: Use 1 ml of buffer per 50 ml of the harvest volume.
9. Rinse the dialysis membrane with DI H₂O
10. Dialyze against 1× Buffer A for 3 times: change every 2 hours, 2 Liter for each change. Last change is for overnight. Add 1 ml NEM and PMSF to dialysis buffer.

Day 3
11. Spin down dialyzed media at 9K for 20 min. Note change in volume.
12. Remove supernatant (51) and put in a separate tube.
13. Resuspend pellet in an equal volume of Buffer B as dialysis volume.
14. Let this sit out on top of bench for about 10 minutes.
15. Centrifuge at 9K for 20 min
16. Remove supernatant (S1′) and place in another tube.
17. Resuspend the pellet in 2 ml of Buffer C centrifuge at 9 k for 20 min and collect supernatant (S2). Type VII collagen will be present in all fractions with different purity. S1 fraction contains approximately 50% type VII collagen but is very dirty. Typically the S1 fraction is not used for further purification. With good dialysis, most will be in S1′ in a purer form. With sub-optimal dialysis, most will be in the S1 fraction, and very dirty. Typically, the S1′ fraction is subject to further Q-sepharose column purification.
   Type VII Collagen Column Purification from S1′.
18. Fill column with sepharose beads ((beads must be shaken into solution before use) and let settle to desired volume.
   The column volume should be approx. ½ loading volume of sample from S1′.
19. The column should not be allowed to dry out. Wash with buffer B 5× column volume (therefore if 4 ml column then wash with 20 ml buffer B)
20. Prepare wash and elution tubes with equal volume as column volume.
21. Save 200 μl of protein sample in small eppendorf and store on ice (to run on gel at end for control)
22. Make and label tubes: 2× wash (buffer B), 0.3M, 0.4M, and 1.0M.
   Everything with a 2× in front of it means 2 times column volume (therefore if column volume is 4 ml then use 8 ml) Load sample to column, being careful not to excessively disturb the column surface. Place tube labeled “flow through” to collect the flow through.
23. Keep everything on ice. When the sample has flowed through once, load the flow through again and collect flow through with tube labeled “flow through.”
24. Before the column runs dry, wash with buffer B (equivalent to sample volume) TWICE and collect with tube labeled wash (therefore if 4 ml then 8 ml buffer B). Continue eluting with increasing salt concentration and ending with 1.0M. 2× wash (buffer B), 2×0.3M, 2×0.4M and 1.0M A, 1.0M B. (Note: most C7 comes out at 1.0 M). Everything with a 2× in front of it means 2 times column volume (therefore if column volume is 4 ml then use 8 ml)
25. Add inhibitors PMSF and NEM. 1:100 fold dilutions to each elution tube (therefore if 40 ml use 40 μl). Most C7 will come out in 0.5-1M eluted fractions.
26. Make samples to run on gel (gel only holds 9 samples at a time). Label 9 small eppendorf tubes×2 (one for western blot and the other for Coomassie staining): load on, follow through, 0.3M, 0.4M . . . 1.0M
27. Make loading dye: 12 μl BME/100 μl of 4× sample buffer→vortex.
28. Add 10 μl of the dye to all tubes.
29. Add 10 ul of sample for ECL, 40 μl for Coomassie Blue to tubes.
30. Run collected fractions on a 6% acrylamide gels for both Western blot analysis and Coomassie Blue staining.

### Concentration and Filtration of Type VII Collagen

1. Combined type VII collagen fractions from 0.5, 0.7 and 1.0 M elution and dilute with buffer B three fold (for example, 17 ml to 50 ml).
2. Load 50 ml of diluted fraction into a 1.5 ml Q-sepharose column two times.
3. Wash column two times with 1.5 ml buffer B.
4. Elute column with buffer B in 1.0 M salt three times (labeling tube as 1.0A, 1.0B and 1.0C).
5. Dialyze the concentrate with PBS.
6. Filter with 0.2 μm Super Membrane Acrodisc Syringe Filter (Pall Life Sciences).

### 7. Store at −80° C. Freezer.

Other embodiments are within the following claims.

1-42. (canceled)

43. A recombinant human collagen 7, or a functional fragment of human collagen 7, made by a method comprising:
providing a cell, which comprises an exogenously introduced nucleic acid that encodes human collagen 7, or a functional fragment thereof, wherein said cell is genetically manipulated to express one or more polypeptides that increase expression of human collagen 7, or a functional fragment thereof, and wherein the one or more polypeptides comprise prolidase;
culturing said cell under conditions sufficient for the production of human collagen 7, or the functional fragment thereof, and prolidase, thereby making human collagen 7, or the functional fragment thereof; and
recovering human collagen 7, or the functional fragment thereof, from said cell culture.

44. The recombinant human collagen 7, or the functional fragment of human collagen 7, of claim 43, wherein said cell is further genetically manipulated to express a glycosyl transferase.

45. The recombinant human collagen 7, or the functional fragment of human collagen 7, of claim 43, wherein said cell comprises an exogenously introduced nucleic acid that encodes prolidase.

46. The recombinant human collagen 7, or the functional fragment of human collagen 7, of claim 44, wherein said cell comprises an exogenously introduced nucleic acid that encodes glycosyl transferase.

47. The recombinant human collagen 7, or the functional fragment of human collagen 7, of claim 43, wherein said cell comprises an expression vector that comprises said exogenously introduced nucleic acid sequence that encodes human collagen 7, or the functional fragment thereof.

48. The recombinant human collagen 7, or the functional fragment of human collagen 7, of claim 47, wherein said expression vector further comprises a nucleic acid sequence that encodes prolidase.

49. The recombinant human collagen 7, or the functional fragment of human collagen 7, of claim 48, wherein said expression vector further comprises a nucleic acid sequence that encodes glycosyl transferase.

50. The recombinant human collagen 7, or the functional fragment of human collagen 7, of claim 43, wherein at least 30, 40, 50, 60, 70, 80, 90 or 95% of said human collagen 7, or the functional fragment thereof, is incorporated into homotrimers.

51. The recombinant human collagen 7, or the functional fragment of human collagen 7, of claim 43, wherein at least 30, 40, 50, 60, 70, 80, 90 or 95% of said human collagen 7, or the functional fragment thereof, is incorporated into hexamers.

52. The recombinant human collagen 7, or the functional fragment of human collagen 7, of claim 43, wherein the exogenously introduced nucleic acid that encodes human collagen 7, or the functional fragment thereof, is a highly glycine codon-optimized nucleic acid sequence.

53. The recombinant human collagen 7, or the functional fragment of human collagen 7, of claim 43, wherein said cell is a mammalian cell.

54. The recombinant human collagen 7, or the functional fragment of human collagen 7, of claim 43, wherein the method further comprises purifying human collagen 7, or the functional fragment thereof, from said cell culture.
